SEQUENCE LISTING (10 sequences)

SEQ ID NO: 1 - DNA, 21 bp, Artificial Sequence
  synthetic
ttaatgcgaccctccgggacg

SEQ ID NO: 2 - DNA, 26 bp, Artificial Sequence
  synthetic
cgcagtacgaggttatttaagtgacg

SEQ ID NO: 3 - DNA, 25 bp, Artificial Sequence
  synthetic
ataatgactgagtacaaactggtgg

SEQ ID NO: 4 - DNA, 27 bp, Artificial Sequence
  synthetic
gtaaatgtagtggtgtgtaccgttagg

SEQ ID NO: 5 - DNA, 24 bp, Artificial Sequence
  synthetic
gcaatgggagccatcgggctcctg

SEQ ID NO: 6 - DNA, 26 bp, Artificial Sequence
  synthetic
taccgtacttggtcggacccgggact

SEQ ID NO: 7 - DNA, 20 bp, Artificial Sequence
  synthetic
tatatgccgggggcgcggcg

SEQ ID NO: 8 - DNA, 24 bp, Artificial Sequence
  synthetic
gccagtcacctgtcctttgcgtgg

SEQ ID NO: 9 - DNA, 26 bp, Artificial Sequence
  synthetic
ataatcctgggcatccacgtcaaacc

SEQ ID NO: 10 - DNA, 25 bp, Artificial Sequence
  synthetic
gccagtcgagtttggacactaaagg

(19) United States
(12) Patent Application Publication
(10) Pub. No.: US 2020/0309773 A1
(43) Pub. Date: Oct. 1, 2020

(54) DISEASE PROTEOME PROTEIN ARRAYS AND USES THEREOF

(71) Applicant: iNanoBio, INC., Tempe, AZ (US)

(72) Inventor: Bharath TAKULAPALLI, Tempe, AZ (US)

(21) Appl. No.: 16/754,872

(22) PCT Filed: Oct. 16, 2018
(86) PCT No.: PCT/US2018/056129

§ 371 Date: Apr. 9, 2020

Related U.S. Application Data
(60) Provisional application No. 62/572,666, filed on Oct. 16, 2017.

(51) Int. Cl. G01N 33/543; G01N 33/574; G01N 33/68

(52) U.S. Cl. CPC G01N 33/54366; G01N 33/6845; G01N 33/57484

(57) ABSTRACT

Provided herein are methods of making and using disease-specific protein arrays. In particular, provided herein are embodiments of disease-specific protein arrays and their use in varied applications such as biomarker detection, diagnostics, elucidating signaling pathways, studying interaction networks and posttranslational modifications, and for drug discovery applications.

### CROSS-REFERENCE TO RELATED APPLICATIONS

This application claims the benefit of U.S. Provisional Application No. 62/572,666, filed Oct. 16, 2017, which is hereby incorporated by reference in its entirety for all purposes.

### STATEMENT REGARDING FEDERALLY SPONSORED RESEARCH

Not applicable.

### BACKGROUND

Functional protein microarrays are an important tool for extracting complex proteomic information from biology. The extracted information can aid in phenotype characterization, correlation with genomics and other omics at the systems level, can aid in early detection of disease, accurate diagnosis and prognosis, precision medicine, objective outcome measures, resolving disease networks and pathways, drug discovery and developing personalized therapeutics. Similar to DNA microarrays, protein microarrays allow for massively parallel screening and analysis of protein interactions with other proteins, nucleic acids, drugs, other biomolecules for high throughput data extraction. However, while functionality of DNA is largely due to the linear sequence of nucleotides, functionality of proteins is determined by three-dimensional polypeptide folding, which can denature rapidly in ex-vivo conditions leading to loss of function.

None of the current methods of producing protein arrays meet the challenges and quality demands for protein-based biosensors. Current protein microarray methods, protein based biosensor technologies when applied to the above applications suffer from many limitations such as low specificity resulting in high false positives, false negatives, low sensitivity of detection, and high signal-to-noise ratios. For example, conventional protein based biosensors use a small array of sensor devices coated with a limited set of predetermined proteins (or other biomolecules) to detect pre-identified biomarker(s) of interest to diagnose a disease. As exemplified by the current controversy over utility of PSA tests, diagnosis of disease based on over-expression or under-expression of a single biomarker (or even a small panel of biomarkers) may lead to sub-optimal decisions in a significant number of cases. Accordingly, there remains a need for improved methods and compositions for detecting the presence of disease-associated proteins, nucleic acids, and other biomolecules, and diagnosing a subject as having a particular diseased based on the results of the detecting.

### SUMMARY

In one aspect, provided herein is a gene variant array comprising a plurality of gene products associated with one or more diseases arrayed on a substrate, wherein each discrete location of the array comprises a target gene product and gene product variants. Each discrete location can comprise a single target gene product and one or more gene product variants. The plurality of gene products can be immobilized at each discrete location as expressed proteins. The plurality of gene products can be expressed in situ at each discrete location of the array by in vitro transcription and translation of target gene nucleic acids and nucleic acids of gene variants obtained from one or more biological samples. The substrate can be selected from the group consisting of a slide, a microwell plate, and a nanowell plate.

In another aspect, provided herein is a method of preparing a gene variant array of this disclosure, the method comprising; (a) providing a first substrate comprising one or more disease-associated biomolecules at one or more discrete locations in an array format; and (b) providing a second substrate comprising an array of biosensors configured to capture the one or more disease-associated biomolecules, wherein the second substrate is in proximity to the first substrate and wherein the array of one or more disease-associated biomolecules is in alignment with the array of biosensors, wherein the array is configured to detect at least one disease-associated target biomolecule in a test sample. The gene variant array can be configured to detect post translational modification of proteins in the array. The gene variant array can be configured to determine kinetic rates of post translational modification.

In a further aspect, provided herein is a method of detecting the presence a target biomolecule in a test sample, the method comprising (a) contacting one or more disease-associated biomolecules to one or more discrete locations in an array format on a first substrate having at least two physically isolated regions; (b) capturing the one or more disease-associated biomolecules at one or more discrete locations on a second substrate to form a monolayer of captured biomolecules in an array format on the second substrate, wherein the second substrate comprises an array of biosensors that capture the one or more disease-associated biomolecules; (c) contacting a test sample to the array of captured biomolecules under conditions that promote binding of target biomolecules to the captured biomolecules if present in the test sample; and (d) detecting binding of target biomolecules to the captured biomolecules at one or more discrete locations on the second substrate, wherein detectable binding indicates the presence of the target biomolecule in the test sample. The one or more disease-associated biomolecules can be proteins expressed by in vitro transcription and translation (IVTT). The array of biosensors on the second substrate can be aligned with the array of one or more disease-associated biomolecules, whereby one or more disease-associated biomolecules is captured directly onto active areas of corresponding biosensors on the second substrate. The active area of a biosensor can be at least one surface in close proximity of a sensor device. At least a portion of biosensors of the array can comprise an electrochemical sensor array, a metal or semiconducting surface, or an insulator surface. The biosensors can comprise quantum dots, nanoparticles, beads, magnetic particles and wherein detection comprises optical detection. The biosensors can comprise calorimetric sensors, potentiometric sensors, SERS (Surface Enhanced Raman Spectroscopy) sensors, amperometric sensors, conductometric sensors, ion channel sensors, ion sensitive sensors, impedance spectroscopy based sensors, or surface-plasmon-polariton sensors, or a combination thereof. The one or more disease-associated biomolecules can be proteins that bind to the second substrate within about 1 nm to about 1 mm of the biosensors. The one or more disease-associated biomolecules can be proteins that bind to directly to at least a portion of a biosensor surface. The proteins can bind using a chemical tag, affinity tag, or covalent binding.

These and other features, aspects, and advantages described herein will become better understood by persons of ordinary skill in the art upon consideration of the following drawings, detailed description, and appended claims.

### BRIEF DESCRIPTION OF DRAWINGS

The invention will be better understood and features, aspects, and advantages other than those set forth above will become apparent when consideration is given to the following detailed description thereof. Such detailed description makes reference to the following drawings.

FIG. 1 illustrates an embodiment of a cancer proteome protein array. Cancer or disease related proteins are arrayed on a substrate for optical dye-based detection, or arrayed on a biosensor surface for detection of protein-sensor interactions. A single chip may comprise proteins that represent all or a subset of wild-type and variant proteins associated with one or more cancers. In this manner, the chip enables multiplexed detection of protein-protein, protein-DNA, and protein-biomolecular interactions between a test sample and a cancer proteome protein array.

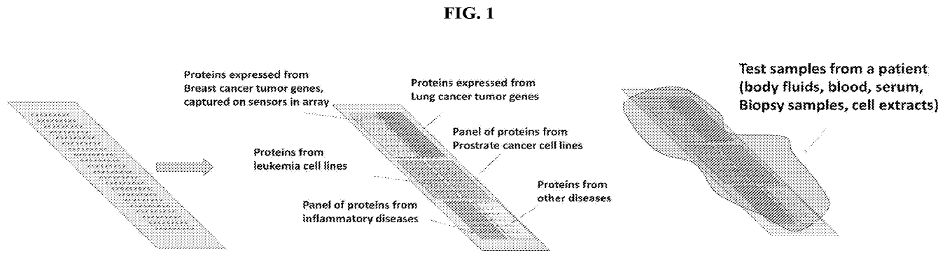

FIG. 2 illustrates use of one or more oligonucleotide primers to isolate variants of genes of interest in a biological sample. Primers can be designed to isolate and further amplify variants including wild-type, alleles, alternate splicing, isoforms, recombinations, polymorphisms and mutated versions of a gene of interest.

FIG. 3 illustrates use of multiple primers to hybridize to and isolate genes of interest in a biological sample. In this embodiment, isolated nucleic acids representing genes of interest separated using a primer are placed in separate wells of a multi-well plate to form a library of cancer-related gene variants.

FIG. 4 illustrates that cancer-related gene libraries can be obtained for biological samples of multiple patients. Alternately, extraction of gene variants can also be done in a single step by first combining biosamples from multiple patients into a single biosample.

FIG. 5 is a schematic representation of an exemplary protocol for extracting nucleic acids of interest for use in a cancer proteome protein array.

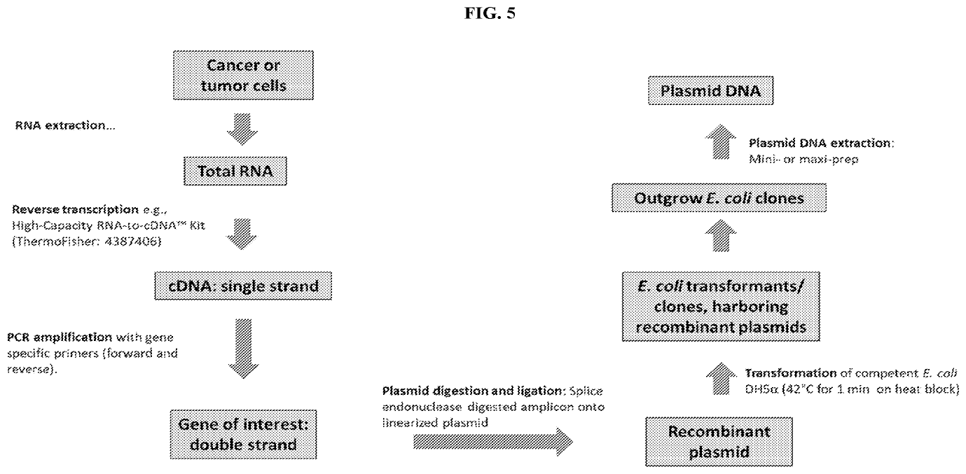

FIG. 6A is a schematic illustrating the isolation of nucleic acids (e.g., DNA, RNA) from a disease biological sample. Isolated nucleic acids can be cloned into expression vectors for in vitro protein expression.

FIG. 6B is a schematic illustrating construction of a disease proteome protein array using nucleic acids isolated in FIG. 6A using, for example, NAPPA or IPC (isolated protein capture) or another protein array technique.

FIG. 6C is a schematic illustrating an exemplary method in which test blood sample comprising antibodies, immune cells are contacted to a disease proteome protein array to obtain an immune signature.

FIG. 7 is a schematic illustrating an embodiment of a cancer proteome protein array.

FIG. 8 is a schematic illustrating an embodiment of a disease proteome protein array comprising NAPPA isolated protein capture (IPC) or contra (cover) capture protein array.

FIG. 9 is a schematic illustrating an embodiment of an array comprising surface capture protein biosensors.

FIG. 10 is a schematic illustrating an embodiment of an array comprising proximity capture protein biosensors.

FIG. 11 is a schematic illustrating an embodiment of an array comprising example electrochemical sensors or field effect sensors or nanowire biosensors and use of the array for antibody profiling of a test sample.

FIG. 12 is a graph demonstrating response of a FDEC charge sensor to SRC kinase auto-phosphorylation by detection of released H⁺. A 200 mV threshold voltage response was produced upon addition of 10 μl of 10 mM adenosine triphosphate (ATP), whereas addition of 10 μl aliquots of pure water and pure adenosine diphosphate (ADP) produced no response.

FIG. 13 is a schematic illustrating detection of acetylcholinesterase interactions using electrode-based sensors.

FIG. 14 is a schematic illustrating detection of kinase phosphorylation using a FET biosensor configured to detect released H⁺.

FIG. 15 illustrates enzymatic activity that can be detected using biosensors described herein.

FIG. 16 illustrates kinds of biosensors appropriate for use in the arrays provided herein for various biosensing applications.

FIG. 17 demonstrates selective binding of cell surface proteins, receptors, or other cell surface molecules to specific proteins in a microarray.

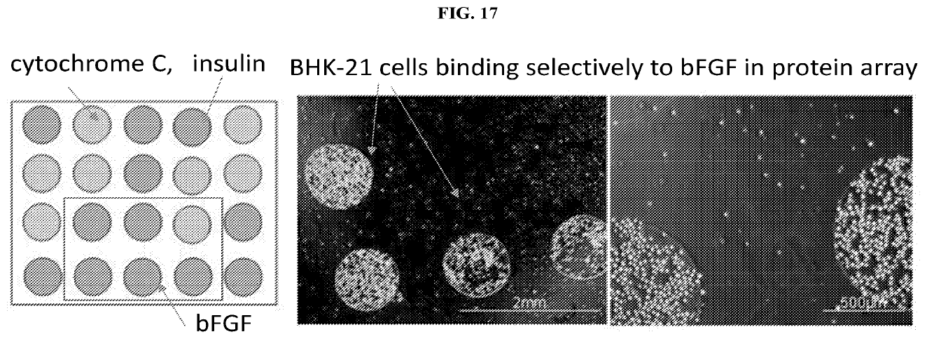

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

All publications, patents, and patent applications mentioned in this specification are herein incorporated by reference to the same extent as if each individual publication, patent, or patent application was specifically and individually indicated to be incorporated by reference.

This disclosure, by way of certain illustrative and non-limiting examples, provides methods and compositions (e.g., proteome protein arrays) for detecting the presence of disease-associated proteins and diagnosing a subject as having a particular diseased based on the results of the detecting. The present disclosure is based at least in part on the inventor's development of protein sensor chips capable of detecting the presence of disease-associated proteins, including disease-associated variant proteins, in a biological sample. In cancers and certain other diseases, a patient's immune system responds to disease by producing antibodies against “foreign” cancer proteins, thus acting as a sentinel of disease. Protein arrays of known cancer proteins can be used to discover subset of these proteins that are immunogenic by profiling for auto-antibodies in serum of cancer patients and comparing with healthy controls. The discovered cancer specific antigens, or the antibodies to these antigens, or combinations of these can then be used as diagnostic and prognostic biomarkers of disease, by way of a simple blood test. Alternately, a disease proteome arrayed on chip (meaning, the protein complement of a tumor or infected tissue) comprising some or all disease-related proteins and their respective mutations arrayed on a single chip can be used for immuno-profiling based diagnosis and prognosis of disease. Furthermore, expressed proteins can be post-translationally modified (PTM), and then assayed with patient serum for identifying antibody or immune response biomarkers against PTM modified proteins, or for accessing protein variant loss or gain in function. Without wishing to be bound by theory, it is believed that disease proteome protein arrays comprising a complement of proteins expressed from genes (wild type, alleles, isoforms, mutations, PTM modifications (native and abnormal) and other variant forms) associated with a disease such as cancer (e.g., derived from or associated with one or more tumors, carcinomas, sarcomas, leukemia, or lymphomas), provide for improved detection methods as well as improved diagnostic and prognostic capabilities for subjects having or suspected of having a disease.

Many current diagnostic tests employ detection of protein biomarkers, which are often present in small numbers. However, antibody, autoantibody, or immune cell responses to the presence of disease or infection can be amplified (e.g., orders of magnitude larger) in a biological sample relative to disease-associated biomarkers themselves. For example, a biological sample may contain biomarkers as well as disease-specific antibodies, but the antibodies are overrepresented by orders of magnitude and can be detected more easily and with higher sensitivity for diagnostic purposes. In this manner, the protein sensor devices provided herein enable one to quickly obtain antibody profiles or immune cell signatures for diagnostic and other clinical purposes using a biological sample such as blood or a tumor biopsy. In addition, by using such a protein sensor platform, it is possible to detect if a tumor is benign or malignant, the tumor type and subtype (e.g., distinguishing between ER+, PR+, and HER2+ samples in the case of breast cancers), the tumor's drug resistance, stage of development, and further detailed molecular sub-typing of cancer. Without wishing to be bound by theory, it is believed that a body's immune response to a particular disease is specific to the type and subtype of disease. For example, benign tumors are expected to have elicit a different antibody response than malignant tumors.

Accordingly, this disclosure provides disease proteome protein arrays (or “disease proteome chip”) and methods of using such arrays for diagnostic and other practical applications. In one aspect of the present disclosure, provided herein is a method of detecting the presence a target biomolecule in a test sample, where the method comprises, or consists essentially of, (a) contacting one or more disease-associated biomolecules to one or more discrete locations in an array format on a first substrate having at least two physically isolated regions; (b) capturing the one or more disease-associated biomolecules at one or more discrete locations on a second substrate to form a monolayer of captured biomolecules in an array format on the second substrate, wherein the second substrate comprises an array of biosensors that capture the one or more disease-associated biomolecules; (c) contacting a test sample to the array of captured biomolecules under conditions that promote binding of target biomolecules to the captured biomolecules if present in the test sample; (d) detecting binding of target biomolecules to the captured biomolecules at one or more discrete locations on the second substrate, wherein detectable binding indicates the presence of the target biomolecule in the test sample.

The terms “proteome protein array” and “proteome chip” are used interchangeably herein and refer sensor arrays coated with proteins or nucleic acids representing all or a subset of naturally occurring human proteins, including proteins having post translational modifications. The proteome protein arrays provided herein can be as an improved alternative to conventional protein microarrays. As used herein, the term “disease proteome” or “disease-ome” refers to sensor arrays coated with unique proteins or antigens associated with one or more diseases. In some cases, the unique proteins or antigens are expressed from genes extracted from a single disease sample (e.g., tumor, cell line, infected cells or tissue) or multiples of tumors/cancers or diseases or other abnormal or infected cells. Likewise, the term “cancer proteome” or “cancer-ome” refers to sensor arrays coated with unique proteins or antigens associated with one or more cancers, including cancer types or sub-types (e.g., including proteins derived from ER+, PR+, and/or HER2+ breast cancer samples).

As compared to conventional protein arrays, which rely on a printed mass of materials and a limited array of sensors, the methods of this disclosure yield improved arrays comprising a large number of sensors for simultaneously detecting a many binding or interacting species, thus minimizing errors such as over or under diagnosis (for p values <0.01). The arrays also advantageously comprise a single monolayer of unique, pure proteins, antibodies, or other biomolecules of interest, directly attached to the surface or in proximity to the sensing element (or multi-layers where a monolayer is not possible).

In some cases, the disease proteome chip comprises a plurality of proteins present at discrete locations (features) on a solid substrate, thereby forming a protein array on the substrate. For the purposes of this disclosure, the term “protein” refers to peptides and polypeptides, including antigens, protein fragments, and modified polypeptides (e.g., proteins having one or more post-translational modifications). While disease proteome arrays of this disclosure are preferably produced using in situ protein expression methods, they can also be produced using other cell based techniques or printing purified proteins. Specific applications of protein biosensors in which proteins are produced in any of these ways are described herein.

In some cases, proteins are immobilized on sensor device surfaces (substrates). In other cases, proteins are immobilized on surfaces in close proximity of one or more sensor devices. In either configuration, the immobilized protein array forms a single sensor chip capable of detecting and diagnosing a unique disease or a set of different diseases, depending on the panels of different disease-associated proteins included in the array.

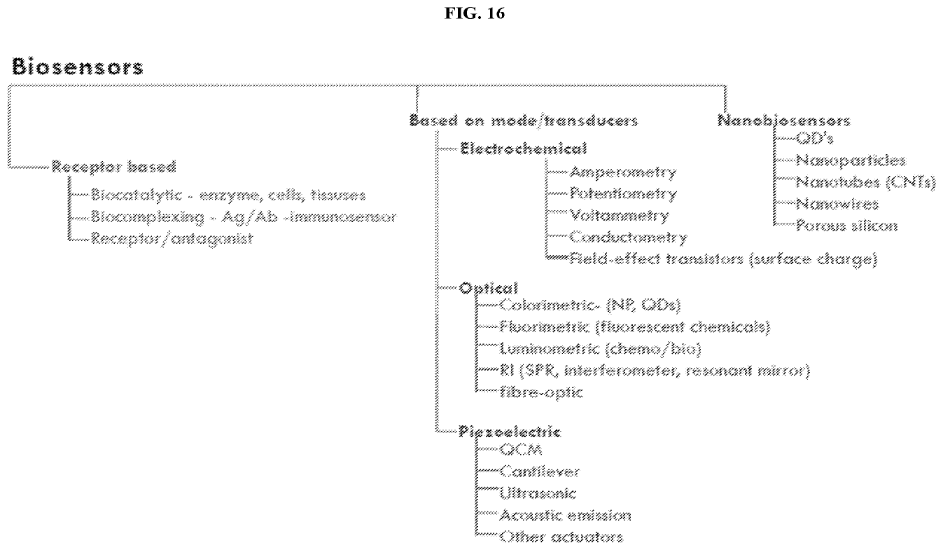

A biosensor is a device that combines a signal transducing (sensing) element with a thin film or chemical or a biological component (biomolecule) to detect, quantify the presence or absence of specific chemical or biomolecular species of interest in a test medium via specific binding, interaction or biochemical reaction. Biosensor arrays are arrays of sensors comprising a unique chemical or biological molecule on each (or multiple) of the sensor units, to combinatorially detect presence or absence of single or multiple biomolecules of interest in a test medium. The signal transducing element can comprise of an optically active tag such as a dye, quantum dot, magnetic particle, nanoparticle, or a radiometric tag. Biosensors can also comprise a sensor device that monitors changes produced in electrical properties such as resistive, capacitive, inductive, or mass, electrochemical, magnetic, plasmonic or magnetic or optical or thermal (or a combination of these) properties of the transducing (sensing) element to detect target chemical or biomolecule of interest. Referring to FIG. 16, examples include, without limitation, field effect transistor (FET) nanowire sensors, ion sensitive FETs (ISFETS), SPR sensors, plasmonic sensors, raman, electrochemical, acoustic sensors, quartz crystal microbalance etc.

As used herein, the term “protein biosensor” is refers to biosensors that sense or detect protein interaction or binding with any other chemical or metabolomics or molecular or biomolecular or ionic species, which in addition can be used to detect kinetics of protein interactions. Provided herein are innovative approaches to coating sensor surfaces (or surfaces in the vicinity/proximity of sensors) with monolayers of in situ expressed proteins, where each sensor in the array is coated with a unique protein monolayer, to yield high density sensory protein arrays for high-throughput assays—capable of in situ time-resolved multiplexed detection of interacting biomolecules with high-sensitivity and high-selectivity. The disease proteome protein array platforms of this disclosure provide for high throughput screening using label-free sensory arrays to solve complex challenges in mining the human proteome, discovering various protein interactions and functions, and can be applied to molecular systems biology in general. Transition from current optical read out methods to methods such as label-free electronic signal readout should bring about advances of similar or greater magnitude as did transition from microwell plates to on-slide high density microarrays.

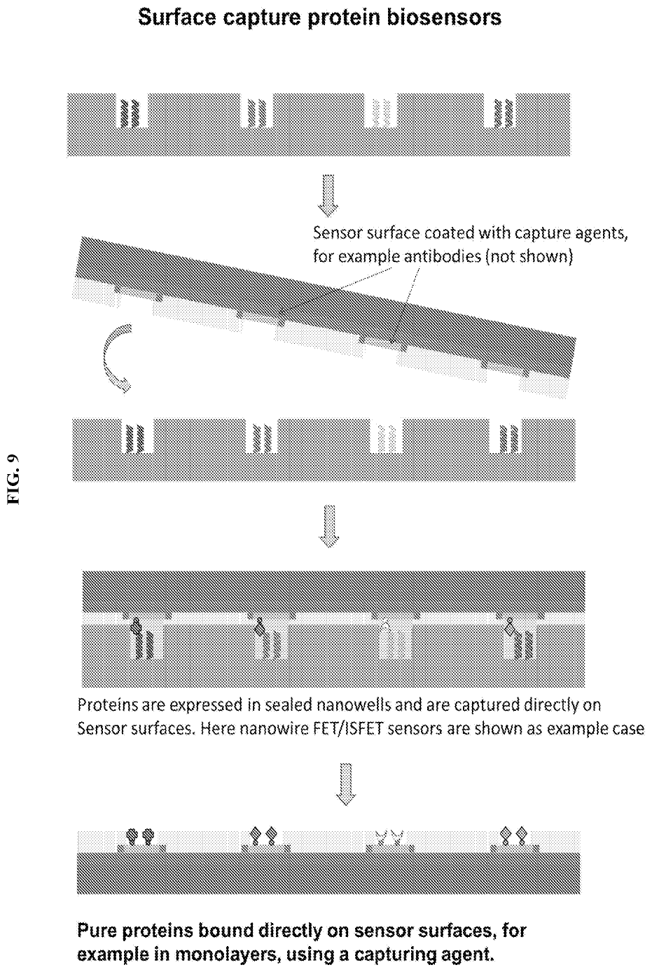

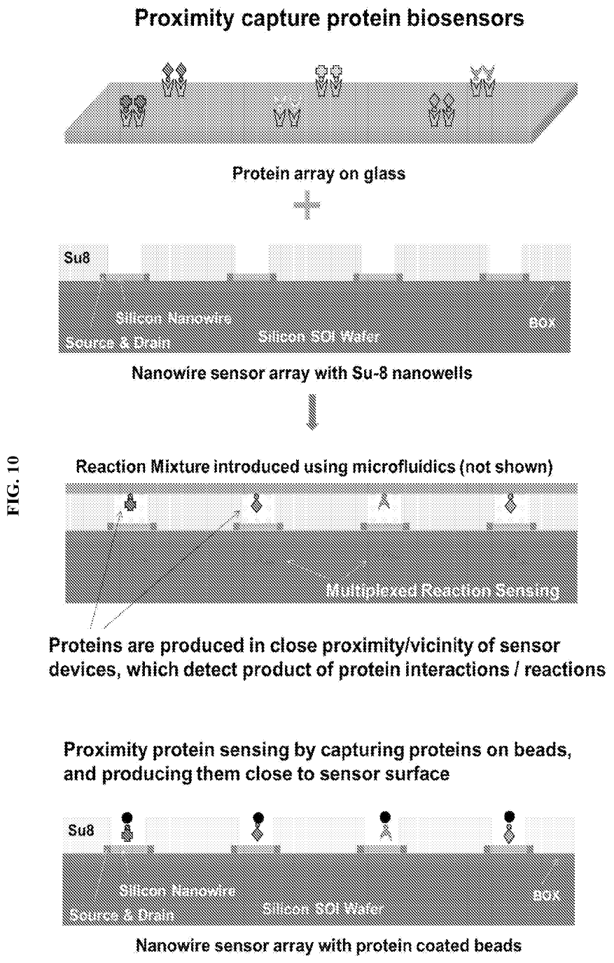

Preferably, protein capture biosensors have one of two configurations: where proteins are coated directly on the surface of sensor devices (direct capture protein biosensors as illustrated in FIG. 9), or alternately, where proteins are coated on a substrate that is in close proximity to sensor devices so that they can sense the products of protein reactions—termed proximity capture protein biosensors. Referring to FIG. 10, the second configuration of proximity sensing protein reaction products suits specific sensing applications where the protein interaction/reaction can be monitored indirectly by detecting the products of the reaction/interaction or a secondary substance in solution.

In some embodiments, “proximity capture protein biosensors” comprise beads or nanoparticles coated with proteins that can be applied on the sensors in the array such that beads in each sensor well have different proteins captured on it. In another configuration, the proximity capture protein biosensor comprises a protein array produced on a second substrate, with array period corresponding to sensor array period, and both the substrates are brought close to each other. In this fashion, each protein on the protein microarray is placed in close proximity (e.g., at a distance of about 1 nm to about 1 mm) to the sensor device.

As used herein, the term “substrate” refers to any type of solid support to which the peptides are immobilized. Examples of substrates include, but are not limited to, microarrays; beads; columns; optical fibers; wipes; nitrocellulose; nylon; glass; quartz; diazotized membranes (paper or nylon); silicones; polyformaldehyde; cellulose; cellulose acetate; paper; ceramics; metals; metalloids; semiconductive materials; coated beads; magnetic particles; plastics such as polyethylene, polypropylene, and polystyrene; gel-forming materials; silicates; agarose; polyacrylamides; methylmethracrylate polymers; sol gels; porous polymer hydrogels; nanostructured surfaces; nanotubes (such as carbon nanotubes); and nanoparticles (such as gold nanoparticles or quantum dots). When bound to a substrate, the proteins can be directly linked to the support, or attached to the surface via a linker. Thus, the solid substrate and/or proteins can be derivatized using methods known in the art to facilitate binding of the proteins to the substrate, so long as the derivitization does not eliminate detection of binding between the proteins and biomolecules that may be present in a test sample.

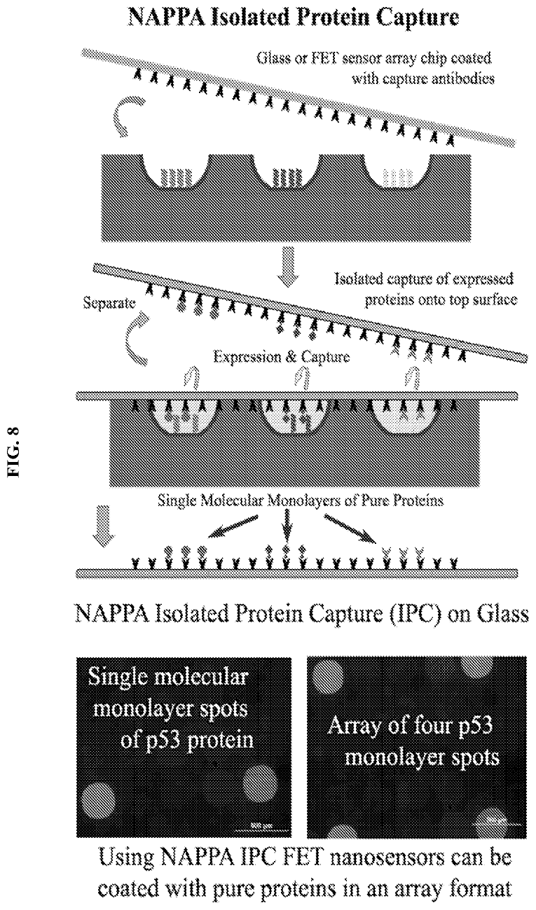

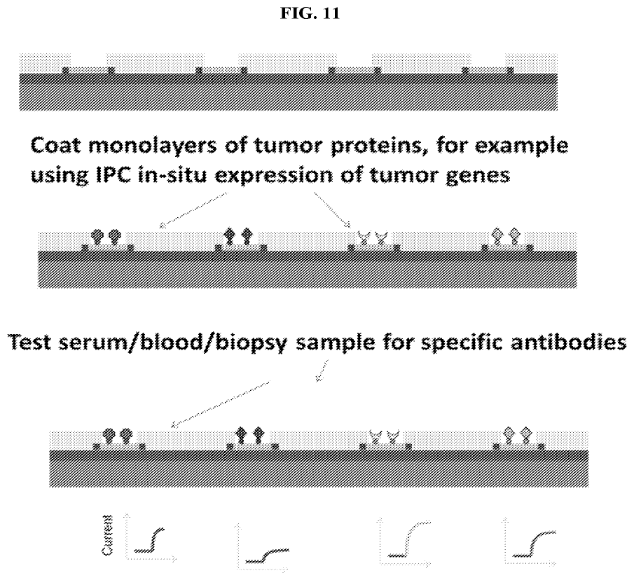

Referring to FIG. 8 and FIG. 11, isolated protein capture procedures can be used to capture monolayers of proteins in an array format onto many different kinds of substrates, such as silicon, silicon dioxide, aluminum dioxide, hafnium oxide (gate dielectrics) and metals such as gold, palladium, by coating their surfaces with capture antibodies. For example, by using a field effect transistor (FET) nanosensor chip comprising of sensor elements in an array with same period corresponding to the period of silicon nanowell substrate, forming a monolayer of capture antibodies (anti-GST) on the device active surfaces, aligning the pattern on the FET sensor chips with nanowell array and press sealing the assembly for isolated protein expression and antibody capture of proteins on devices—it is possible to coat each sensor in the array with monolayer of a unique protein—a breakthrough advance enabling sensory protein arrays. The FET sensory protein arrays thus produced with self-assembled protein monolayers (or multi layers) on active nano-sensor surfaces can be used for high-sensitivity high-selectivity time-resolved electronic-detection of interactions with other proteins and biomolecules. Another exemplary method of coating sensor arrays with different proteins is using cell-based protein synthesis methods, or by printing prior purified proteins on unique devices.

In some embodiments, the protein is provided by transcribing and translating a nucleic acid molecule provided at a discrete location on the sensor substrate, or on a substrate in close proximity to the sensor substrate. In this manner, proteins of the array are either produced on the sensor substrate (FIG. 9) or are produced in close proximity to the sensor substrate (FIG. 10). In such cases, disease-associated nucleic acids are deposited at discrete locations on the array and the disease proteome protein array is expressed in situ using, for example, cell-free in vitro transcription and translation reagents. For such in situ generated protein arrays, nucleic acids such as cDNA, genes, or plasmids are printed on a substrate (e.g., a glass substrate, silicon nanowells) and incubated with in vitro transcription and translation (IVTT) mixture to express fresh proteins, right at the point of use. While it is possible to coat a limited array of sensors with monolayers of pure proteins that have been prior expressed and purified, it is not possible to do this for large array of sensors with tens of thousands of proteins without loss to protein functionality. Current state of art in protein based biosensors use a small array of sensor devices coated with a limited set of predetermined proteins (or other biomolecules) to detect pre-identified biomarker(s) of interest. Accordingly, in situ generated protein arrays are particularly advantageous for large arrays of sensors (e.g., about 100 sensors up to 100,000 sensor units), where each sensor is coated with monolayer of a unique, pure proteins, antibodies, or other biomolecules of interest. As used herein, the term “antibody” refers to immunoglobulin molecules and immunologically active portions (fragments) of immunoglobulin molecules, i.e., molecules that contain an antibody combining site or paratope. The term is inclusive of monoclonal antibodies and polyclonal antibodies.

In some embodiments, a protein is deposited at one or more discrete locations on the substrate, thus forming a protein array on the substrate. For example, prior-expressed purified proteins can be printed at discrete locations on an array substrate.

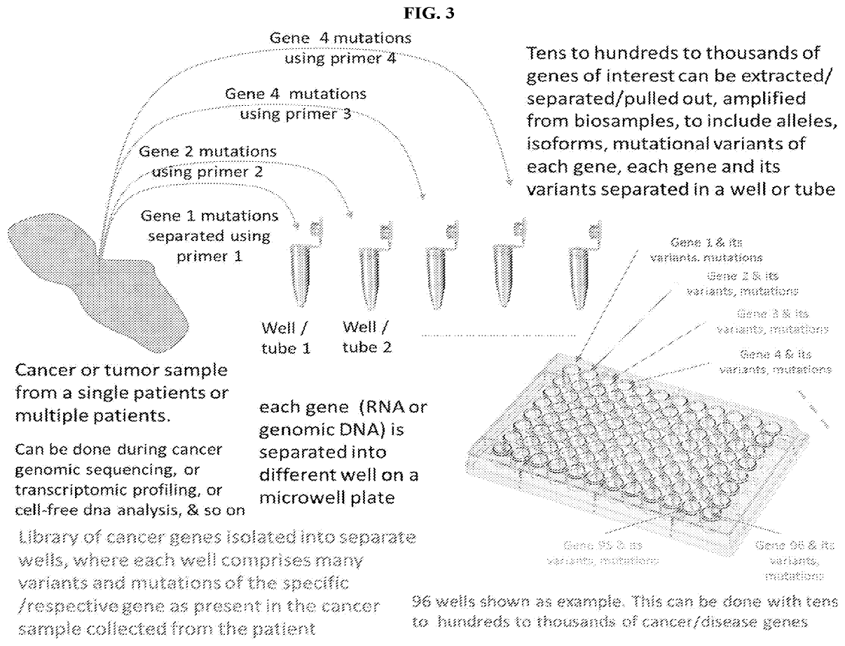

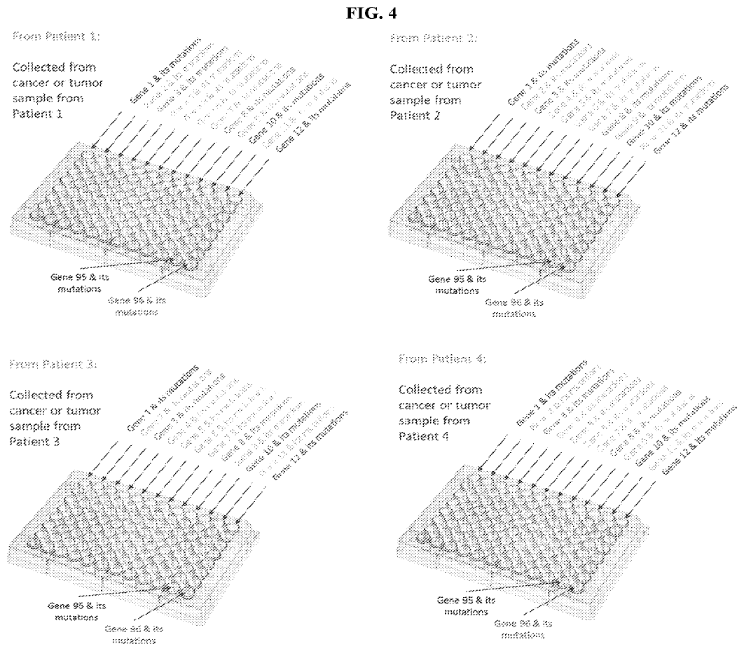

As illustrated in FIGS. 3 and 4, in some cases the chip is prepared using nucleic acids obtained from a biological sample (e.g., a cancer sample, tumor biopsy sample). The nucleic acids can be RNA, DNA, e.g., genomic DNA, mitochondrial DNA, viral DNA, synthetic DNA, or cDNA reverse transcribed from RNA. The nucleic acids in a nucleic acid sample generally serve as templates for extension of a hybridized primer. In a preferred embodiment, nucleic acid molecules are isolated from a biological sample. By contacting one or more oligonucleotide primers having complementarity to a nucleic acid sequence of interest (e.g., a gene of interest) under conditions that promote hybridization of the oligonucleotide time primers to complementary nucleic acids, one may select and isolated nucleic acids corresponding to RNA or DNA of a gene of interest. Contacting oligonucleotide primer(s) to nucleic acid molecules from a biological sample can occur prior to or after performing an amplification reaction to amplify the number of copies of a nucleic acid sequence of interest. In the case of RNAs of interest, contacting oligonucleotide primer(s) to nucleic acid molecules from a biological sample can occur prior to or after performing a reaction to convert the RNA to cDNA. In some cases, nucleic acid molecules isolated from a total nucleic acid sample can be used for producing a chip without further processing. In other cases, the isolated nucleic acid molecules can be amplified or modified in some way prior to placement on a chip.

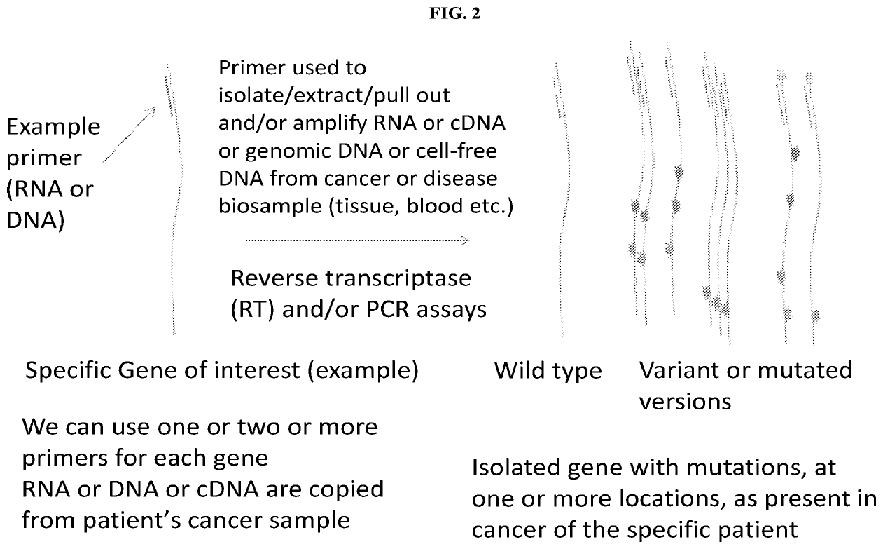

When a gene of interest is separated (e.g., isolated) from a biological sample (e.g., tumor sample) using a primer having a length of about 10-100 nucleotides or more (e.g., about 10, 20, 30, 40, 50, 60, 70, 80, 90, 100 nt), or, in some cases, having a length of a few hundred or thousand nucleotides, mutational versions of specific genes present in the sample will be isolated along with wild-type copies. Referring to FIG. 2, in this way, separating nucleic acid sequences from a mixed nucleic acid sample using one or more primers will simultaneously isolate wild-type copies as well as any mutational versions of a gene of interest present in the sample. The terms “isolated” or “purified” refer to material that is substantially or essentially free from components which normally accompany the material as it is found in its native state.

As used herein, the term “variant” refers to an alteration in the normal sequence of a nucleic acid sequence or an amino acid sequence (e.g., a gene or a gene product). In some instances, a genotype and corresponding phenotype is associated with a variant. In other instances, there is no known function of a variant. A variant can also mean a sequence difference relative to a reference sequence. A variant can be a single nucleotide polymorphism (SNP). A variant can be an insertion of a plurality of nucleotides. A variant can be a deletion of a plurality of nucleotides. A variant can be a mutation. A variant can be a copy number variation. A variant can be a structural variant.

Any appropriate oligonucleotide amplification method can be used according to the methods described herein. Polymerase chain reaction (PCR) is a process for amplifying one or more target nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. The skilled artisan to detect the presence of desired sequence (U.S. Pat. No. 4,683,195) routinely uses polymerase chain reaction. A specific example of PCR that is routinely performed by the skilled artisan to detect desired sequences is reverse transcript PCR (RT-PCR; Saiki et al., Science, 1985, 230:1350; Scharf et al., Science, 1986, 233:1076). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan.

The use of primers to extract nucleic acid sequences of interest is well known to those in the art and methodologies are available. In some cases, oligonucleotide primers are in solution. In other cases, oligonucleotide primers are bound to beads, particles, magnetic particles, a surface of a well-plate, or slides.

Any appropriate method can be used to isolate nucleic acids from a biological sample such as a tissue or tumor biopsy. For example, the sample can be treated with solutions that lyse cells within the sample and precipitate nucleic acids.

As used herein, the term “sample” means non-biological samples and biological samples. Non-biological samples include those prepared in vitro comprising varying concentrations of a target molecule of interest in solution. Biological samples include, without limitation, blood, lymph, serum, urine, saliva, sputum, breath extract (meaning exhaled air captured in a solution), bone marrow, aspirates (nasal, lung, bronchial, tracheal), eye fluid, amniotic fluid, feces other bodily fluids and secretions, cells, and tissue specimens and dilutions of them. Any suitable biological sample (“biosample”) can be used. For example, a biological sample can be a specimen obtained from a subject (e.g., a mammal such as a human, canine, mouse, rat, pig, guinea pig, cow, monkey, or ape) or can be derived from such a subject. A subject can provide a plurality of biological samples, including a solid biological sample, from for example, a biopsy or a tissue. In some cases, a sample can be a tissue section or cells that are placed in or adapted to tissue culture. A biological sample also can be a biological fluid such as urine, blood, plasma, serum, saliva, tears, or mucus, or such a sample absorbed onto a paper or polymer substrate. A biological sample can be further fractionated, if desired, to a fraction containing particular cell types. In some embodiments, a sample can be a combination of samples from a subject (e.g., a combination of a tissue and fluid sample). In some cases, sera are obtained from the individual using techniques known in the art. The sample may be any cell sample potentially harboring the target protein(s) or other biomolecule(s) of interest. For example, a cytology sample may be obtained from a tissue selected from breast, ovaries, esophagus, stomach, colon, rectum, anus, bile duct, brain, endometrium, lung, liver, skin, prostate, kidney, nasopharynx, pancreas, head and neck, kidney, lymphoma, leukemia, cervix, and bladder. The sample may be a solid or non-solid tumor specimen. The tumor specimen may be a carcinoma. The sample may be a new cancer, recurrent cancer, primary cancer, or metastasized (secondary) cancer.

The sample may be obtained by methods known in the art, such as surgery, biopsy, or from blood (e.g., circulating tumor cells), ascites, or pleural effusion. The sample may be processed using methods known in the art. For example, the sample may be fresh, frozen, or formalin-fixed and paraffin-embedded (FFPE).

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. Thus, in addition to being useful for human diagnostic, prognostic or predictive applications (e.g., diagnosing a disease in a human patient), the methods and devices of the present invention may also be useful for veterinary treatment of mammals, including companion animals.

The terms “cancer” and “malignancy” are used herein interchangeably to refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer. Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas.

As used herein, the term “tumor” refers to all neoplastic cell growth and proliferation, whether malignant or benign, and all pre-cancerous and cancerous cells and tissues. For example, a particular cancer may be characterized by a solid mass tumor or non-solid tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography) or magnetic resonance imaging (MRI), or by needle aspirations.

Making Arrays of Disease Genes from Variants of Key Genes Extracted and/or Amplified from a Patient Biosample

This section provides an exemplary work flow for producing a disease proteome array from nucleic acids extracted from a patient biological sample (“biosample”). While this example discusses arrays prepared for human patient biosamples, the methods are equally applicable for samples obtained from other animals or even plant biosamples.

1. Acquire patient biosample: In some cases, the biosample is obtained from a patient known to have a particular disease such as cancer. Suitable samples are tissues (e.g., biopsy sample), blood, and other biosamples.

2. Biosamples used for an array can correspond to a specific type, sub-type, or stage of disease: Biosamples can be from one patient or can be combined biosamples from multiple patients. In one example, the tissue is obtained from a patient having stage 1 breast cancer. Other samples: tissue from triple negative breast cancer; combination of tissues collected from multiple different patients, each having stage 1 breast cancer; combination of tissues collected from multiple different patients, each having primary lung cancer of different stages; combination of tissues collected from multiple different patients, each having metastatic lung cancer; blood collected from one or more leukemia patients; saliva, blood, urine or other biosample collected from patients having other diseases such as diabetes, autoimmune disorders, neurodenegerative disease, etc.

3. For each cancer, there may be a few to tens, hundreds, or thousands of key genes that play a role. Key genes can be over expressed or under expressed, can carry mutations, or can be alleles, or polymorphisms, or isoforms or alternatively spliced variants and in some cases, proteins expressed from key genes are post translationally modified (normal or abnormal disease related or random PTMs)—all of which can be called variant proteins for the specific gene.

4. For each biosample, one or more primers designed to specifically hybridize to a specific key disease gene (e.g., cancer key gene) are used to extract and, in some cases, amplify wildtype and variants of the key gene present in the biosample. In some cases, the primers are gene-specific primers complementary to RNA in a tissue sample in a reverse transcriptase assay and/or followed by PCR amplification, whereby the reaction extracts key gene including variants to which the gene-specific primers correspond. In other cases, the primers are gene-specific primers complementary to genomic DNA (e.g., DNA extracted from the nucleus, chromatin, chromosomes), where the primers extract key genes and variants to which the gene-specific primers correspond. In some cases, the primers are designed to extract extra-nuclear DNA or cell-free DNA (e.g., found in circulating blood). Starting with one or a few primers specific to a key gene of interest, the assay can be replicated for a few to tens, hundreds, or thousands of key genes of interest associated with a disease or cancer.

5. Each gene variant is collected in a separate well of a microwell plate, a separate tube, a separate nanowell, or some suitable spot or location or vessel.

6. With the extracted DNA variants, it is possible to create arrays for each disease of interest (e.g., an array for lung cancer, breast cancer, prostate cancer, neurodegenerative disease, etc.).

Producing Array Products

In this section, we describe various array products that can be made using gene variants collected in Example 1. For the purposes of this disclosure, the term “array” encompasses microarrays, nanoarrays, and arrays prepared on slides or microwell plates or nanowell substrates. Biomolecular variants in the array may be captured or otherwise immobilized to a surface using a capture molecules, or biomolecular variants can be in solution in discrete locations (e.g., wells) of a microwell or nanowell plate.

In some cases, the array is a gene variant array. These arrays comprise RNA or DNA variants dispensed or spotted in an array format. Each spot can have one key gene and its variants extracted from patient samples as described above. The array format can be a microwell plate, microarray slide, or a slide comprising an array of nanowells. Gene variant arrays are useful (a) as a repository of transcriptomic (RNA) or genomic (DNA) variants for further analysis; (b) or studying DNA variant interactions with other biomolecules and cells; (c) for gene expression analysis (d) for analysis of mutational load; (e) for PCR based amplification and detection of biomarkers; and (f) for gain of-function or loss-of-function analysis (g) for combining with complementary proteomic analysis for higher accuracy disease diagnosis, prognosis and precision medicine.

In another embodiment, the key genes (and variants of each) are cloned into expression vectors (e.g., plasmids). The vectors or genes are used to express proteins in in-vitro transcription and translation (IVTT) systems, cellular expression systems, or using phage display. Express protein microarray with each well or spot comprising many variants of a key protein that have been expressed from corresponding key gene variants cloned into plasmids.

In a further embodiment, the key genes are fused with a common epitope tag, and the combination gene-epitope tag fusion construct is cloned into an expression vector (e.g., plasmid). The plasmids are printed to discrete locations (spot, nanowell, etc.) and are expressed in situ using IVTT, or are expressed in a cell-free or cellular system. The common tag is used to capture the expressed proteins, using a common anti-epitope binding ligand or antibody immobilized on same surface or a secondary surface. For expressed protein microarrays, in which each spot comprises many variants of a key protein that have been expressed from corresponding key gene variants, the expressed proteins are captured or otherwise immobilized on a solid surface.

In some cases, the anti-epitope binding ligand or antibody or binding agent is immobilized on same surface (e.g., NAPPA protein array or IPC isolated protein capture). In other cases, the anti-epitope binding ligand or antibody or binding agent is immobilized on a second surface. The second surface may be glass or another type of surface, and detection is achieved using fluorescence, luminescence, or radiometric methods, or other tag based methods. The second surface can be a biosensor array surface, where biosensors may be FETs, SPR, GMR, raman, or nanotube or nanowire sensors, plasmonic graphene, or any other sensors (FIG. 16). Other detection methods used may be mass spectroscopy-based methods, Matrix Assisted Laser Desorption Ionisation (MALDI) or Surface Enhanced Laser Desorption Ionization (SELDI) TOF, laser or liquid chromatography, HPLC based methods, tandem MS or TIMS (Thermal Ionization-Mass Spectrometry), AMS (Accelerator Mass Spectrometry), ICP-MS (Inductively Coupled Plasma-Mass spectrometry). In some cases, the second surface may be nanoparticles or magnetic particles or other beads or micro particles.

For some arrays, gene variants are fragmented into smaller DNA strands using methods well known to those in the field of art. Gene fragments for all variants are expressed as respective peptide variants for each of the key genes at each spot. As another example, protein variants of each key protein/gene are expressed and then fragmented into peptides using enzymatic, chemical, mechanical, or other methods known to practitioners in the art.

In some cases, protein variant arrays may be expressed in situ, as described above. Alternately, proteins of a protein variant array are expressed prior to forming the array and then deposited or printed in an array format. In this manner, the proteins are provided as products for subsequent assays. In such cases, the protein variant array does not require in situ protein expression via IVTT and can be used as an off-the-shelf product. For example, key protein variants can be produced in larger quantities from respective key gene variants. Many different key proteins can be produced at a manufacturer's facility and the key protein variant array is produced by spotting or printing proteins in an array format on an appropriate substrate (e.g., on a slide, on a microwell plate, on a nanowell slide). By way of example, HuProt Arrays are printed protein arrays prepared in this fashion.

Protein Variant Arrays for Post-Translational Modifications (PTM): Protein variant array produced in the above methods (from gene variants extracted from cancer/disease patient biosamples) is post translationally modified (PTM), using some or all of enzymes, co-factors, chemicals, biochemicals, solutions or a combination of these, to produce natural (wild type) or disease related or abnormal or random PTMs. For example, a specific kinase or few kinases along with co-factors and other assay components can be used to phosphorylate proteins on the protein variant assays. The variants of each protein can have varying propensity to PTM modification, which in turn may cause a differences in interactions with other proteins, DNA, drug molecules, which may cause loss of function or gain of function.

Performing Assays Using Protein Arrays

This section describes assays that can be performed using the array products described in Example 2. In some cases, assays are performed to detect interactions of variant protein arrays with other biomolecules (e.g., other proteins, antibodies, DNA, RNA, small molecules, chemicals etc). Such assays are useful for research purposes, diagnostic or prognostic purposes, for drug discovery purposes, for therapeutic development purposes, for disease network discovery, for target identification, or for immunotherapy development. Modes of detection for assays can be (i) fluorescence or luminescence or radiometric or other labeled/tagged detection assays; (ii) FET or SPR or graphene or plasmonic or magnetic or electrochemical sensor based or other biosensor based detection assays or label-free detection assays (FIG. 16); (iii) mass spectroscopy or liquid chromatography based methods (such as TOF MALDI/SELDI), or a combination of these. Express protein arrays with each spot or well comprising many variants of a key protein that is expressed from corresponding key gene variants. The arrays can be expressed in-situ at the time of assay for follow-on applications. Additional exemplary assays are described below:

Assays for Biomarker Discovery: Protein variant microarray produced in the above methods/devices, is screened with serum or tissue lysate or cell lysate or blood or other biosamples from (i) cancer/disease patients (ii) healthy controls, to detect interactions with proteins, antibodies, dna, rna, biochemicals and so on in these secondary biosamples—to identify specific cancer/disease biomarkers. Biomarkers as used here can be for early detection, diagnosis, prognosis, disease monitoring, precision medicine, personalized medicine, disease pathway specific biomarkers, pathogenesis, pathway/network identification biomarkers, clinical endpoint biomarkers, outcome biomarkers

Assays for Antibody Profiling Signaturing: For these assays, test samples are screened using protein variant microarrays produced for a specific disease. Test samples are preferably serum, blood, a tissue lysate, or cell lysate from test individual. The presence of antibodies in the test sample that bind to one or more proteins of the protein variant array indicates that the test individual may have the specific disease as described above. In some cases the protein variant array is produced using cancer-specific key genes and their variants, extracted from one or more patients that have the specific cancer. For example, a lung cancer protein variant array can be used to detect and diagnose lung cancer in test individuals. A lung cancer protein variant array can comprise of sub-arrays of protein variant arrays specific to pre-stage 1 lung cancer, stage 1 lung cancer, stage 2 lung cancer, stage 3 lung cancer, stage 4 lung cancer, and so on. If a test individual's results show a larger number of antibodies to stage 2, then the test individual has a likelihood of having stage 2 lung cancer. A protein variant array can be developed comprising sub-arrays corresponding to each specific cancer, such as prostate cancer, lung cancer, brain tumors, pancreatic cancer, breast cancer, ovarian cancer, leukemias, melanoma and so on, and may further include sub-sub-arrays for each specific stage and specific sub-types in each of the cancers.

Immune Cell Assays or Cellular Assays. Immune cells isolated from blood or cells extracted from tissue samples or other biosamples can be screened using disease/cancer protein variant arrays for identifying disease related protein variants. The immune cells can be T cells, B cells, natural killer cells, regulatory cells, memory cells, macrophages, Granulocytes, Mast cells, Monocytes, Dendritic cells, Neutrophils or other immune-related cells. Cell surface receptors, MHCs (major histocompatibility complex), g-protein coupled receptors, enzyme linked receptors, ion-channel coupled receptors, hormonal receptors, integrins, growth factor receptors, neural receptors, cell surface proteins, lipids, glycans, lectins, adhesins or other biomolecules, or receptors such as PAMP receptors, TLRs, NLRs, pattern recognition receptors (PRR), killer activated and killer inhibitor receptors (KARs and KIRs), complement receptors, Fc receptors, B cell receptors and T cell receptors, NK cell receptors on immune cell surfaces may interact with epitopes arrayed on a protein variant arrays. Screening for and detecting such interactions is useful for disease diagnosis or prognosis of the disease associated with the protein variant array. Alternately, screening can be done using, Stem cells, Red blood cells (erythrocytes), White blood cells (leukocytes), Platelets, Nerve cells (neurons), Neuroglial cells, Muscle cells (myocytes), Cartilage cells (chondrocytes), Bone cells, Skin cells, Endothelial cells, Epithelial cells, Fat cells (adipocytes), Sex cells (gametes) or cells from other tissues to detect interactions between proteins of the protein variant array and cell surface receptors, cell surface proteins, or other cell surface biomolecules found in the test sample, or with their respective cell extracts post lysis.

Gain-of-Function and Loss-of-Function Assays: Preferably, a disease/cancer variant protein array comprises many variants for each key protein at each spot or well. In many cancers/diseases, protein variations lead to gain of function or loss of function relative to wild-type proteins. Variant protein arrays can be tested using gain- or loss-of-function assays (using optical light-based or biosensor-based detection methods) to identify key proteins that play role in dysregulation or dysfunction leading to pathogenesis, disease networks/pathways, metastasis, late stage development, and so on.

Assaying Disease Genotype-Phenotype Correlations: The assay can comprise sequencing key genes and their variants and correlating the results with variant protein array assay results, to achieve deep genotype phenotype correlations. Such correlations are important for elucidating disease signaling pathways and disease pathogenesis, for identifying biomarkers for early disease detection, ford disease diagnosis and prognosis, for precision medicine, on related clinical and research applications.

Data Analysis

Data collected in performing the assays described in Example 3 (e.g., biomarker detection for disease prognosis or diagnosis, for monitoring for biomarker changes pre- or following administration of a therapeutic) can be analyzed by a variety of analytical techniques. Exemplary data analysis methods include, without limitation, detecting specific biomarkers in test individual, detecting at least a few biomarkers from a larger set of possible biomarkers in test individual. For example: A cancer/disease may have 50 biomarkers. One test individual may have at least 5 of these 50 possible biomarkers to indicate the presence of a disease. Another test individual may carry a different set of at-least 5 biomarkers from the possible 50, which also might be sufficient criteria for disease diagnosis. In some cases, dynamic combinatorial biomarker analysis, permutational and combinatorial analysis for signature analysis, potentially using dynamic ROC curves, from data generated using advanced data mining, neural networks, machining learning, and artificial intelligence based algorithmic approaches, potentially using cloud computing, for large-scale, high-throughput patient screening and validation data. Data analysis methods may be such as, but not limited to, those discussed in “Deep learning for computational biology” Christof Angermueller et all, Molecular Systems Biology (2016) 12, 878 and “Genomic, proteomic, and metabolomic data integration strategies, Kwanjeera Wanichthanarak et al, Biomarker Insights 2015:10(S4), and data analysis for normalization, pattern recognition, time-series analysis, cross-omics comparisons and multiple-hypothesis testing discussed/included in “Perseus and MaxQuant software platforms” (coxdocs.org/doku.php on the World Wide Web) which are included here in whole by reference.

Primer Design

This section illustrates an exemplary method for designing gene specific primers, preferably from most conserved regions on the coding parts of a gene, to extract target genes of interest and variants thereof. Designing the primers on the most conserved regions of the gene results in increased number of gene variants extracted from patient biosamples. For gene-specific primer design for cDNA synthesis, one must use mRNA translation sequence of the gene of interest. The genomic DNA sequence contains introns that are spliced during RNA processing to yield mRNA, and hence the primers may be designed from the exon regions when starting from mRNA. When using genomic DNA (gDNA), post-processing to make genes accessible for transcription, key genes of interest may be first transcribed to RNA and then reverse-transcribed to cDNA, which may then be amplified. Alternately, post processing of genomic DNA to make genes accessible for PCR using methods known to those in the field of art (example: remove nucleosomal proteins using methods such as phenol/chloroform purification cycles, or using other chemical and/or enzymatic treatment or fragmentation methods), gene variants can be directly copied and amplified from genomic DNA. When extracting/copying/amplifying gene variants from genomic DNA, primer design from most conserved exon regions may be preferred, if it is desired to also extract/copy/amplify RNA from the biosample.

Sequence of a gene of interest is generated in silico using serial cloning as shown below. By way of example, we selected sequences for primers specific to human epidermal growth factor receptor (EGFR). EGFR is a transmembrane protein that is a receptor for members of the epidermal growth factor family (EGF family) of extracellular protein ligands. A forward primer was designed for complementarity to sequence at the 5′ end of EGFR's open reading frame from the mRNA translation. The reverse primer was designed based on sequence at the 3′ end of EGFR's open reading frame. Specifically, the reverse primer represents the reverse complement of the antisense or lower strand from the 3′ end. In the following examples, bold nucleotides indicate those selected for inclusion in each primer. Nucleotides that do not appear in bold can be added to the primer sequence, for example, to achieve different Tm values for the two primers (difference ˜5° C.). A suitable Tm calculator is provided at promega.com/a/apps/biomath/index.html?calc=tm on the World Wide Web.

Forward EGFR primer: 5′-TTAATGCGACCCTCCGGGACG-3′ (SEQ ID NO:1) Tm=61° C.

Reverse EGFR primer: 5′ CGCAGTACGAGGTTATTTAAGTGACG 3′ (SEQ ID NO:2) Tm=58° C.

Another reverse primer: 5′-ATAATCCTGGGCATCCACGTCAAACC 3′ (SEQ ID NO:9) Tm=61° C.

Another reverse primer 4: 5′ GCCAGTCGAGTTTGGACACTAAAGG 3′ (SEQ ID NO:10) Tm=60° C.

Primers were selected for target gene NRAS from mRNA translation sequence or cDNA. The NRAS gene provides instructions for making a protein called N-Ras that is involved primarily in regulating cell division. A forward primer was designed for complementarity to sequence at the 5′ end of human NRAS's open reading frame from the mRNA translation. The reverse primer was designed based on sequence at the 3′ end of the human NRAS open reading frame. Nucleotides that do not appear in bold can be added to the primer sequence, for example, to achieve different Tm values for the two primers.

Forward NRAS primer: 5′-ATAATGACTGAGTACAAACTGGTGG-3′ (SEQ ID NO:3) Tm=55° C.

Reverse NRAS primer: 5′-GTAAATGTAGTGGTGTGTACCGTTAGG-3′ (SEQ ID NO:4) Tm=58° C.

Primers were selected for target gene ALK from mRNA translation sequence or cDNA. The ALK gene provides instructions for making a protein called ALK receptor tyrosine kinase which transmits signals from the cell surface into the cell through a process called signal transduction. A forward primer was designed for complementarity to sequence at the 5′ end of human ALK's open reading frame from the mRNA translation. The reverse primer was designed based on sequence at the 3′ end of human ALK's open reading frame. Nucleotides that do not appear in bold can be added to the primer sequence, for example, to achieve different Tm values for the two primers.

Forward ALK primer:
Tm = 66° C.
(SEQ ID NO: 5)
5′-GCAATGGGAGCCATCGGGCTCCTG-3′
Reverse ALK primer:
Tm = 66° C.
(SEQ ID NO: 6)
5′-TACCGTACTTGGTCGGACCCGGGACT-3′

Primers were selected for target gene BRAF from mRNA translation sequence or cDNA. The BRAF gene provides instructions for making a protein called B-RAF. B-RAF protein is part of the RAS/MAPK signaling pathway, which regulates the growth and division (proliferation) of cells, the process by which cells mature to carry out specific functions (differentiation), cell movement (migration), and programmed cell death (apoptosis). A forward primer was designed for complementarity to sequence at the 5′ end of human BRAF's open reading frame from the mRNA translation. The reverse primer was designed based on sequence at the 3′ end of human BRAF's open reading frame. Nucleotides that do not appear in bold can be added to the primer sequence, for example, to achieve different Tm values for the two primers.

Forward BRAF primer:
Tm = 67° C.
(SEQ ID NO: 7)
5′-TATATGCCGGGGGCGCGGCG-3′
Reverse BRAF primer:
Tm = 65° C.
(SEQ ID NO: 8)
5′-GCCAGTCACCTGTCCTTTGCGTGG-3′

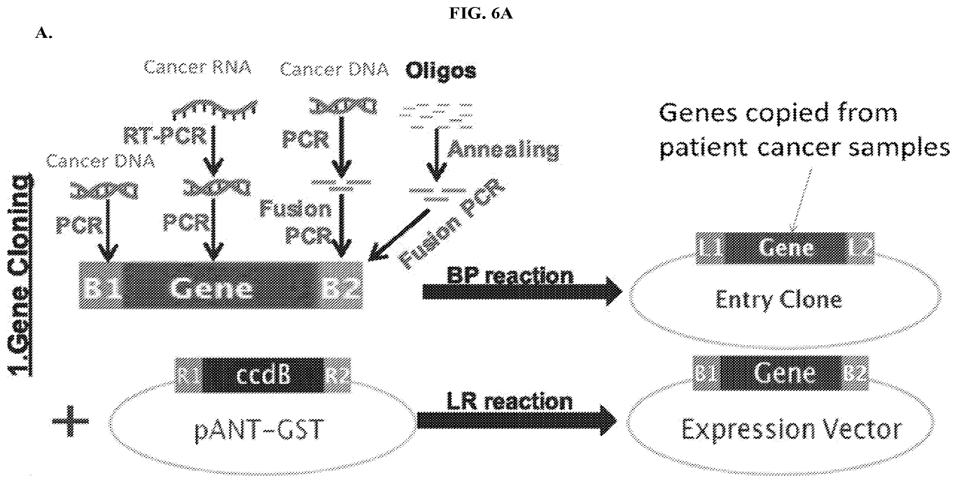

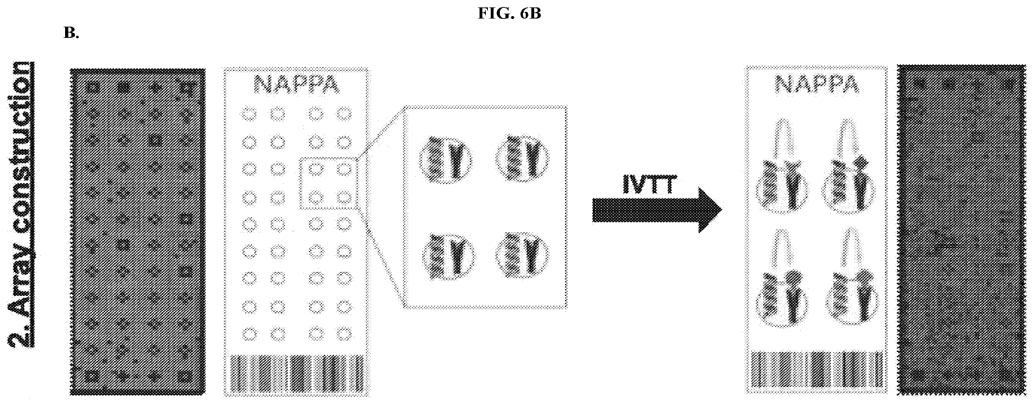

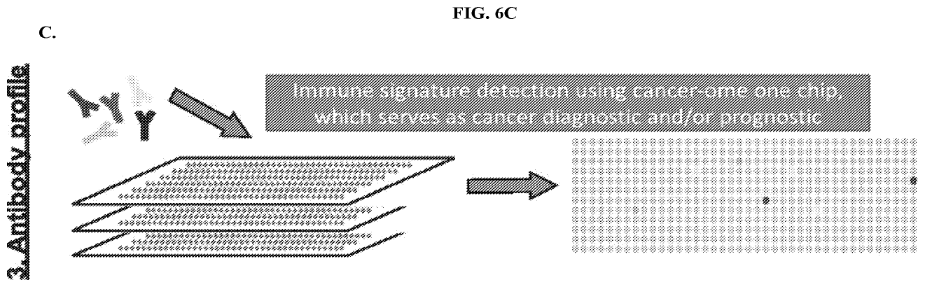

In some cases, genes collected are used to produce protein microarrays using any of the available methods: using ex situ protein micro array methods or in situ protein micro array methods. For example, protein microarray technology that can be used includes, without limitation, Nucleic acid programmable protein array (NAPPA) (see FIG. 6B), or IPC (isolated protein capture) (see FIG. 8), Protein in situ array (PISA), In situ puromycin-capture, DNA array to protein array (DAPA), Nanowell protein arrays, analytical microarrays (also known as capture arrays), functional protein microarrays (also known as target protein arrays), and reverse phase protein microarray (RPPA).

A protein sensor array comprising anywhere from a few sensors up to millions of sensors in the array can be functionalized using any appropriate protein coating technique known in the art such as, for example, NAPPA or IPC.

In some cases, an antibody signature or cell based immune response, which means a binding pattern of antibodies or a cell based immune response to proteins, mutant variant proteins, or proteins having post-translational modifications, is detected by ELISA or similar methods, by optical dye scanning (e.g., optical dye tag based detection with dyes such as FITC, CY3 dyes), or using biosensors. In some cases, an antibody signature or cell based immune response is detected by coating disease proteome proteins on a biosensor surface, which can then be used to detect protein interactions with high sensitivity and specificity in multiplexed format, for diagnostic or prognostic screening, or personalized therapy development. Use of label free biosensors yields kinetic binding data which improves diagnostic and prognostic data quality by reducing the incidence of false positive and false negative results. As used herein, the term “bind” refers to any physical attachment or close association, which may be permanent or temporary. The binding can result from hydrogen bonding, hydrophobic forces, van der Waals forces, covalent, or ionic bonding, for example.

By way of example, this section describes one application of a disease proteome protein array of this disclosure. A biological sample acquired from test patient can be contacted to or mixed with magnetic nanoparticles or beads coated with anti-human secondary antibodies (usually used as secondary antibodies, prepared in any host) for a brief period of time. This results in binding of all antibodies present in the test fluid to be captured on to the magnetic particles, which can then be separated out from test fluid in multiple wash steps. Alternately, the magnetic beads can be coated with antibody capture agents such as chemical linkers, mix&go coating, bio-conjugates etc. Antibodies captured on the magnetic particles can then be chemically or enzymatically separated from the magnetic particles, and the resulting pure antibody solution can be assayed with protein sensor array chips to detect antibodies specific to the disease, thus aiding disease diagnosis and prognosis. Alternately, magnetic particles comprising captured antibodies can be directly assayed on protein sensor array chips, and the multiplexed signals from sensor arrays can be used to detect and diagnose diseases and other human conditions.

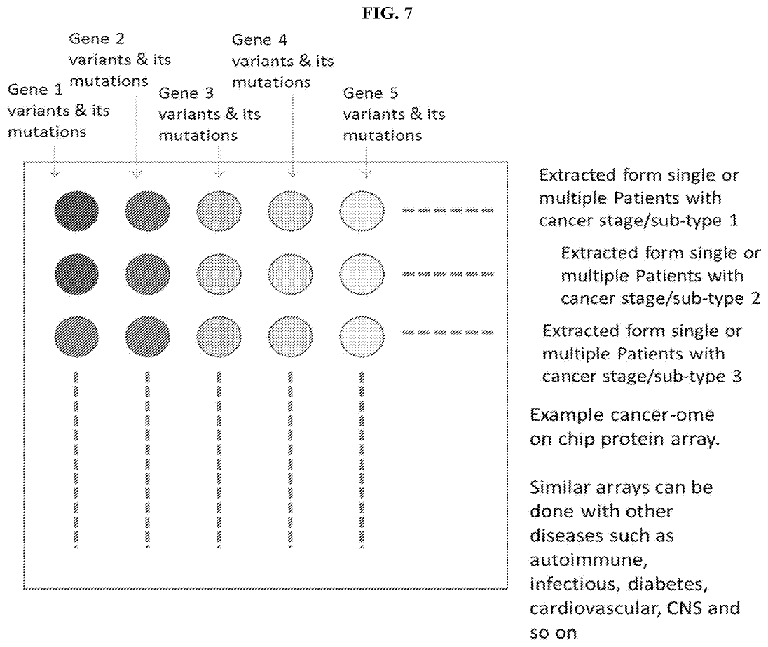

As illustrated in FIG. 7, the protein array can be a cancer proteome protein array comprising cancer-associated genes and their mutant variations as well as genetic information associate with a particular cancer subtype or stage. For example, a blood or serum sample collected from a subject can be assayed on a cancer proteome (“cancer-ome”) chip. If present in the sample, immune response markers such as tumor-specific or other disease specific antibodies bind to one or more of cancer-associated protein spots (antibodies might be generated against mutated proteins and wild type proteins) on the cancer-ome chip, indicating that the subject is likely to have the type of cancer associated with the protein spots. The detected optical dye screening signature or biosensor array signature is data that can be analyzed using bioinformatics for disease diagnosis, prognosis, drug discovery, drug resistance profiling and/or monitoring, and drug interaction profiling.

If the detected pattern is similar to that observed in patients having a particular type of cancer, then the test patient has high probability of having that cancer. By qualifying and quantifying the antibody signature, the size of tumor and stage of disease can also be determined, because larger numbers of antibodies are often present for later stage cancers as compared to early stage cancers. Disease proteome protein arrays can be performed in an afternoon for diagnostic or prognostic purposes. In some cases, the array is performed to detect the presence or absence of cancer in a patient. It can be used as a screening test, diagnostic test, prognostic test for one or many types of cancer by including proteins related to each of the cancer types.

In some cases, disease proteome protein arrays are frozen and stored or shipped for on-demand use, for example, at a different location (e.g., at a clinic, in the field). In some cases, disease proteome proteins arrays are lyophilized for storage and/or shipment for use in a different location.

In some embodiments, the methods provided herein can be used to produce disease proteome protein arrays modified with proteins or antigens expressed from genes of any or all disease-causing pathogens such as virus, bacteria, fungi, Protozoa, helminths, prions, and other single and multi-cellular diseasing causing agents. Since the immune system of a patient who is infected with a disease-causing agent will produce antibodies against the disease-causing agents or components thereof, profiling of the antibody signature produced specifically in response to the infectious agent can serve as an ideal diagnostic to detect infection in a patient. Protein biosensors produced by expressing proteins from pathogens can hence be used to detect antibody response of test patient to detect and diagnose infection, contraction, development of specific diseases. Such sensors can be used in clinical applications (e.g., diagnosing infections) and also in biodefense applications to detect agents of biological warfare, pandemic infections, etc. In addition to humans, the methods and specific applications described in the disclosure can be used to detect and diagnose diseases, infections and conditions in other animals, including wild animals, pet animals, livestock, etc.

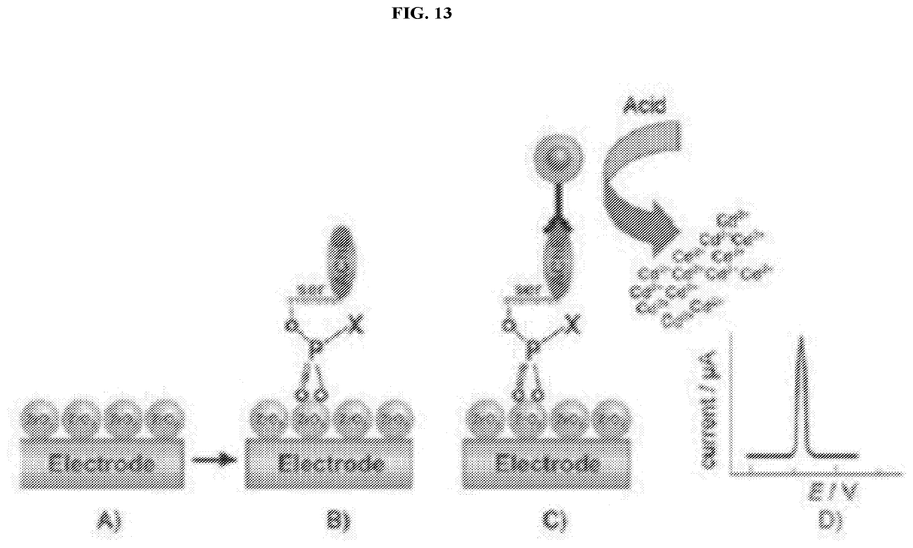

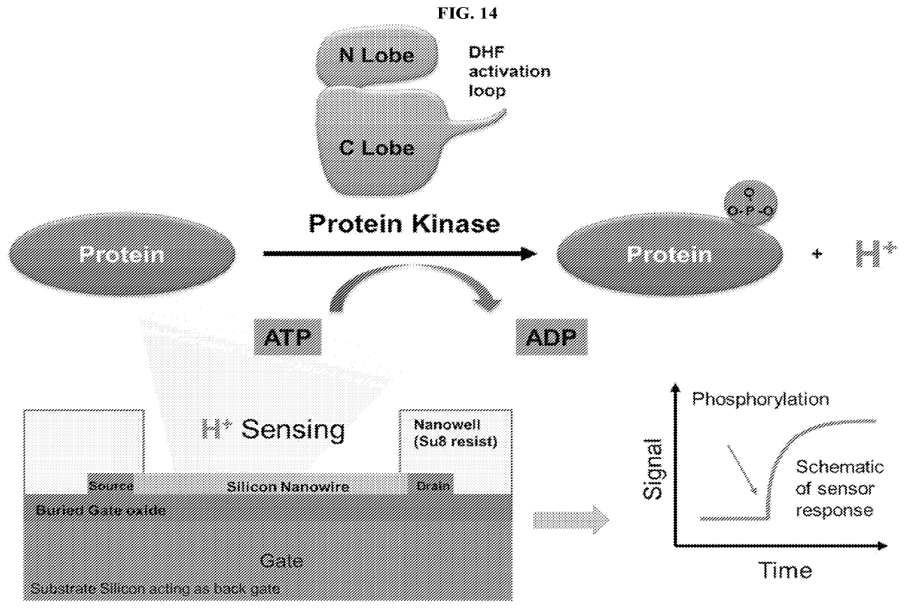

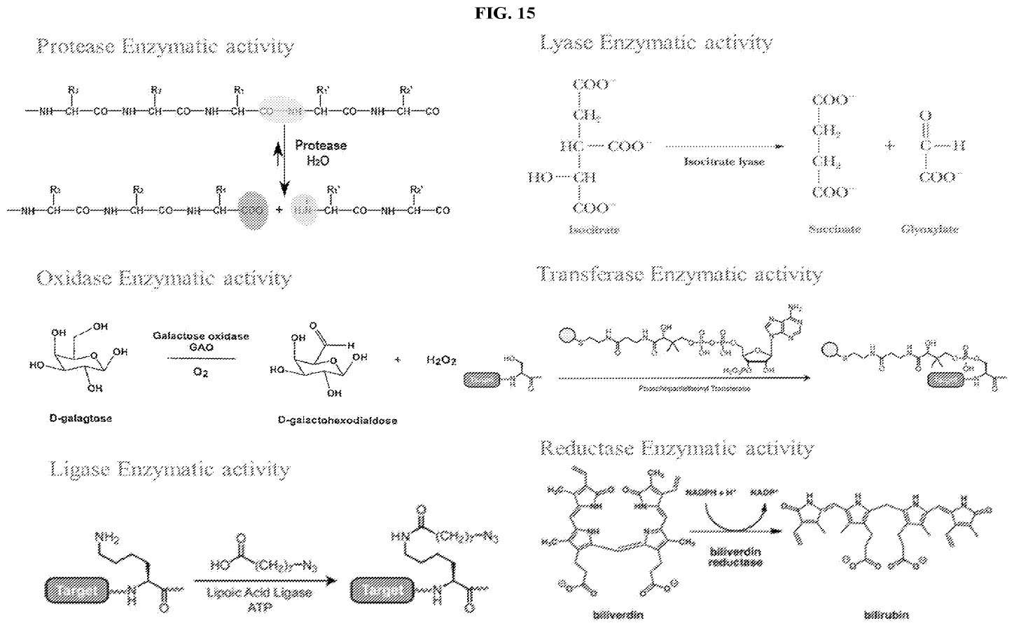

In another aspect, provided herein is a method for using a disease proteome protein array chip to detect enzymatic activity. For example, at least one enzyme of interest can be added to sensor-bound protein arrays and specific activity of the enzyme against the panel of proteins present can be detected via the sensor response. In another example, enzyme proteins produced and captured on sensor locations. The resulting enzyme biosensors can be used to detect specific activity against test proteins, DNA, or other biomolecules in a test sample. In both cases, the enzymes are either (i) directly bound to sensor surfaces to detect changes to enzymes produced by their reactions or (ii) they react with target molecules and the reaction products are detected by the sensors. In some cases, biosensors are configured to detect electrons or protons produced by enzyme-catalyzed reactions. In other cases, biosensors are configured to detect products of an enzyme-catalyzed reaction. Exemplary enzymatic reactions that can be detected using biosensors are illustrated in FIG. 13, FIG. 14, and FIG. 15.

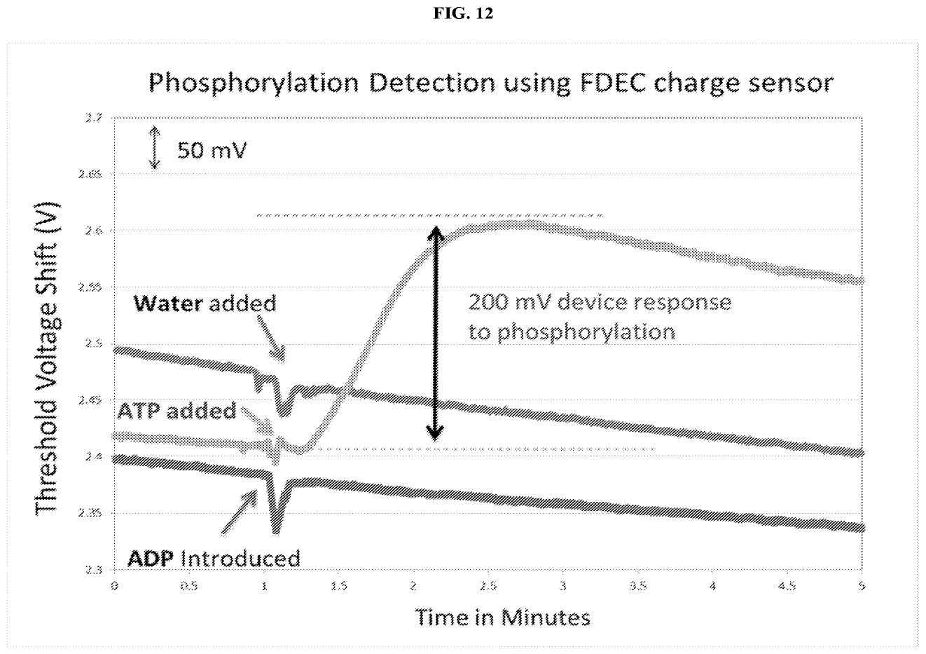

In a further aspect, protein biosensors described in this application can be used to detect post-translational modifications (PTM) of sensor array-bound proteins. As shown in FIG. 12, a FDEC charge sensor can be used to detect SRC kinase auto-phosphorylation by detection of released H⁺. PTMs that can be detected include, but are not limited to, acylation, acetylation, de-acetylation, formylation, alkylation, methylation, amidation, glycosylation, oxidation, glycation, phosphorylation, biotinylation, ubiquitination, SUMOylation, Neddylation, sulfation, pegylation, citrullination, dephosphorylation, deamidation, and eliminylation.

Protein variant arrays of a cancer/disease described herein may be screened with blood from a specific patient carrying the cancer/disease for immunophenotyping, to identify a set of variant proteins in the protein array that are immunogens producing antibodies in the specific patient, or that produce cell mediated immune response involving either T-Cell or B-Cell or NK Cell or other immune cells involving any of TCR, TLRs, NLR, KARs, KIRs, PAMP, PRR or MCHs or other immune cell surface receptors or surface proteins or biomolecules. The protein variant array screening assays enable identifying a broad set of antigenic protein variants in the specific patient, detected using antibody profiling assays or immune cell binding assays performed on protein variant arrays. Following a general immune-phenotyping assays described here, Mass spectroscopy-based or liquid chromatography-based methods such as SELDI or MALDI TOF can be used to identify specific variant (allele, isoform, mutation, PTM modification) of a protein that is immunogenic or antigenic. Further using label free biosensor assays of protein variant arrays can be used to resolve the relative affinities or interaction strengths or binding kinetics of board set of antigenic protein variants in the specific patients, to rank and down select the broad antigenic variant protein set to a sub-set of few to few tens or few hundreds of high-affinity or optimally immuno-interacting antigenic variant proteins. For example, when screening cancer patient blood (or other biosample) with the relevant cancer protein variant arrays, 200 (a large number) of patient specific antigenic protein variants may be identified. Using label-free biosensor assays (such as SPR, FET sensors, raman, plasmonic, electrochemical sensors), kinetic screening can be performed to down select to top 10 or top 25 or top 100 high-affinity or optimally immune-interacting antigens in the specific patients. The immunogens or antigens may be called neoantigens or auto-antibodies or tumor-specific antigens (TSAs). Antibodies or immune-cell surface receptors sensitive to these antigenic protein variants in the specific patient, may then be used to develop more optimal cell-based immunotherapy personalized to the specific patient (based on immunogens discovered in the patient), to improve cancer immunotherapy outcomes, as described in “Neoantigens in cancer immunotherapy” Ton Schumacher et al, Science 3 Apr. 2015; “Tumor neoantigens: building a framework for personalized cancer immunotherapy”, Matthew Gubin et al, J Clin Invest. 2015 September; 125(9); “Dendritic Cell-Based Immunotherapy: State of the Art and Beyond”, Kalijn F. Bol et al, CCR Focus, Volume 22, Issue 8, pp. 1897-1906 2016; Example methods of developing immunotherapies based on identified cancer or patient tumor specific immunogens or neoantigens or TSAs are as discussed in “Driving gene-engineered T cell immunotherapy of cancer”, Laura A Johnson & Carl H June, Cell Research volume 27, 2017.

Example cell-based immunotherapies used may be, but not limited to, CAR-T, T-Cell, B-Cell, NK cell, dendritic cell, other immune-cell based immunotherapies. For example, the identified patient specific variant antigens may be used to optimally design chimeric antigen receptors (CARs) or engineered T cell receptors (TCRs) for T-Cell immunotherapy. The identified immunogens may also be used to improve outcomes of checkpoint immunotherapy, for example by guiding optimal design of inhibitor therapy.

In another aspect, cancer antigens identified using cancer proteome protein arrays of this disclosure can be used to develop personalized neoantigen cancer vaccines as prophylactic anti-cancer agents or as personalized immunotherapy for cancer patients. Two recent studies reported success of personalized immunogenic neoantigen vaccines in melanoma. See Ott et al., Nature 547:217-221 (13 Jul. 2017); Sahin et al., Nature 547:222-226 (13 Jul. 2017). Individual mutations identified by extracting cancer-associated target genes and variants thereof from biological samples according to methods described herein can be exploited for personalized immunotherapy for patients with cancer.

In another aspect, provided herein are method for treating a subject based on their antibody signature or immune profile determined using a disease proteome protein array of this disclosure. The terms “treating”, “treatment” and “therapy” are used herein to refer to curative therapy, prophylactic therapy, and preventative therapy. The terms embrace both preventative, i.e., prophylactic, and palliative treatments. Thus, in the context of the present disclosure the term “treating” encompasses curing, ameliorating or tempering the severity of neuronal loss, necroptosis, and/or associated diseases or their symptoms. In some cases, the term “treated” refers to any beneficial effect on progression of a disease or condition. Beneficial effects can include reversing, alleviating, inhibiting the progress of, preventing, or reducing the likelihood of the disease or condition to which the term applies or one or more symptoms or manifestations of such a disease or condition. “Preventing” or “prevention” means preventing the occurrence of the necroptosis or tempering the severity of the necroptosis if it develops subsequent to the administration of the compounds or pharmaceutical compositions of the present invention. The term “inhibit” is used to describe any form of inhibition that results in prevention, reduction or otherwise amelioration of neuronal loss associated with necroptosis. As described herein, inhibition of neuronal loss includes both complete and partial inhibition of neuronal loss or necroptosis. In one embodiment, inhibition is complete inhibition. In another embodiment, inhibition is partial inhibition.

In the context of this disclosure, the term “administering” and variations of that term including “administer” and “administration”, includes contacting, applying, delivering or providing a compound or composition of the invention to a subject by any appropriate means. For example, in the context of administering an agent that is an inhibitor of neuronal loss or necroptosis activation to a subject, an effective amount of an agent is, for example, an amount sufficient to achieve a reduction in neuronal loss as compared to the response obtained without administration of the agent.

The methods described herein can be carried out using a computer programmed to receive data (e.g., data from a disease proteome protein array indicating whether a subject has an immune profile/antibody signature associated with a particular cancer). The computer can output for display information related to a subject's biomarkers or immune profile/antibody signature. A professional (e.g., medical professional) can communicate information regarding proteome protein array analysis to a subject or a subject's family. In some cases, a professional can provide a subject and/or a subject's family with information regarding particular disease therapy, including treatment options and potential side effects. In some cases, a professional can provide a copy of a subject's medical records to communicate information regarding biomarker analysis and/or disease states to a specialist.

A professional (e.g., research professional) can apply information regarding a subject's biomarkers to advance research into anti-cancer therapeutics or treatment regimens for other diseases. For example, a researcher can compile data on the presence of a particular antibody profile with information regarding the efficacy of a particular therapy, or side effects associated with particular therapy. In some cases, a research professional can obtain a subject's biomarker information to evaluate the subject's enrollment, or continued participation in a research study or clinical trial. In some cases, a research professional can communicate a subject's biomarker information to a medical professional, or can refer a subject to a medical professional for clinical assessment and/or treatment.

Any appropriate method can be used to communicate information to another person (e.g., a professional), and information can be communicated directly or indirectly. For example, a laboratory technician can input biomarker information into a computer-based record. In some cases, information can be communicated by making a physical alteration to medical or research records. For example, a medical professional can make a permanent notation or flag a medical record for communicating information to other medical professionals reviewing the record. Any type of communication can be used (e.g., mail, e-mail, telephone, and face-to-face interactions). Information also can be communicated to a professional by making that information electronically available to the professional. For example, information can be placed on a computer database such that a medical professional can access the information. In addition, information can be communicated to a hospital, clinic, or research facility serving as an agent for the professional.

Articles of Manufacture

This disclosure also provides articles of manufacture that can include, for example, materials and reagents that can be used to determine whether a subject has an antibody profile or immune response profile associated with a particular disease (e.g., cancer). An article of manufacture can include, for example, disease-associated nucleic acids, or polypeptides immobilized on one or more substrates (e.g., in discrete regions (“features”) with different populations of isolated nucleic acids or polypeptides immobilized in each discrete region). The article of manufacture can also include instructions for use in practicing a method for predicting the likelihood of a subject as having a particular disease as provided herein.

The article of manufacture may further comprise one or more disease proteome protein arrays for performing the analysis. In some cases, nucleic acid or protein arrays are attached to a solid substrate, e.g., a porous or non-porous material that is insoluble. The nucleic acids or proteins of each array can be immobilized on the substrate covalently or non-covalently.

Also provided are kits containing any of the disease proteome protein arrays described herein. The kits can optionally contain instructions for detecting one or more antibody profiles or immune response signatures described herein. The kits can optionally include, e.g., a control biological sample.

In some cases, one or more reagents for processing a biological sample and/or using the arrays (e.g., reducing reagents, denaturing, deglycosylating reagents, dephosphorylating reagents, alkylating reagents and/or reagents for chemically or enzymatically cleaving a peptide or protein) are provided with the kit. A kit also can include a detection reagent for detecting the presence or absence of a particular signature. Alternatively, such reagents may be provided separately from the kit.

Instructions for the above-described articles of manufacture are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g., via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The kits described herein also can optionally include instructions for treating a cancer patient based on the presence or absence of an antibody profiles or immune response signatures as described herein.

The terms “determining”, “measuring”, “evaluating”, “assessing,” “assaying,” and “analyzing” can be used interchangeably herein to refer to any form of measurement, and include determining if an element is present or not. These terms can include both quantitative and/or qualitative determinations. Assessing may be relative or absolute. “Assessing the presence of” includes determining the amount of something present, as well as determining whether it is present or absent.

The articles “a” and “an” are used herein to refer to one or to more than one (i.e., to at least one) of the grammatical object of the article. By way of example, “an antibody” means one antibody or more than one antibody. As such, the terms “a” (or “an”), “one or more,” and “at least one” are used interchangeably herein.

It is contemplated that any embodied method or composition described herein can be implemented with respect to any other method or composition described herein.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

While the present invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed. To the contrary, it should be appreciated that many equivalents, alternatives, variations, and modifications, aside from those expressly stated, are possible and fall within the spirit and scope of the invention as defined by the appended claims.

The invention will be more fully understood upon consideration of the following non-limiting Examples.

### EXAMPLE

### Example 1—Protocol for Cloning Gene Mutations from RNA Extracted from Biological Samples [Prophetic]

A. RNA Extraction

To separate the total RNA from other cellular components, the cancerous cells/tissues may be lysed to release its contents, followed by a series of centrifugation steps in TRIzol Reagent. Total RNA includes all mRNA, transfer RNA, ribosomal RNA, and other noncoding RNAs. As desired, mRNA may be selectively extracted from the total RNA using a commercial mRNA extraction kit. mRNA can also be extracted directly from the cell or tissue lysate without an initial total RNA extraction. Many commercial kits for direct mRNA extraction from tissue lysate are available.

mRNA Extraction:

The mRNA isolation kit includes Oligo (dT)₂₀ primer that binds directly to the poly adenylated tail of mRNA, enabling isolation of mRNA via the polyA tail. The isolated mRNA may be used directly for reverse transcriptase assay for cDNA synthesis to obtain variants of gene of interest present in patient sample. The cDNA may then be spliced onto a cloning plasmid to yield a recombinant plasmid, which can then be used to transform E. coli DH5alpha.

For free circulating RNA or DNA (e.g., in human serum or plasma), cell disruption is not required. We may simply spin down the sample at low speed and then perform nucleic acid extraction, using commercial kits. While nucleic DNA or cytoplasmic RNA require cell lysis followed by centrifugation, cell-free DNA/RNA requires initial centrifugation only.

B. cDNA Synthesis

After isolation of mRNA, cDNA may synthesized by reverse transcription from mRNA to DNA using gene-specific primers which essentially recognize a portion of the mRNA. We were interested in “isolating” the gene of interest (and its mutations) from the rest of the polycistronic mRNA. Our ultimate goal was to isolate the gene of interest and its mutation and generate recombinant plasmids for cloning and purification.

C. Gene-Specific Primer Design for cDNA Synthesis

Assuming mutations did not affect sequences at the 5′ and 3′ ends of the gene of interest (GOI), universal primer pair can be designed based on the those two ends. This primer pair should basically synthesize cDNA with the different gene mutations. If however the mutation affected sequences at either one of the 5′ or 3′ ends of the GOI, then it will be best to design a primer pair based on DNA sequence that are adjacent to the GOI on the genomic DNA.

D. Generation of Recombinant Plasmid (Ligation)

Another set of primers which has restriction enzyme recognition sequences at the appropriate ends will be designed and then used to PCR amplify the above cDNA. The recognition sequence to be used for the new primers shall depend on the restriction enzymes on the cloning plasmid. The PCR product will be purified by gel electrophoresis and then digested with the appropriate restriction enzymes. The circular cloning plasmid will also be linearized with the same restriction enzymes followed by ligation of the linearized plasmid and digested PCR products to yield circular recombinant plasmids that harbor the genes/mutations of interest.

E. Transformation of E. coli

The recombinant plasmid prepared as described above may be used to transform E. coli DH5alpha using electroporation or heat shock method. Transformed E. coli may be grown in a suitable medium to yield more E. coli cells. The cells may subsequently be pelleted and then lysed to extract the recombinant plasmid, which may then be used for NAPPA or IPC (isolated protein capture).

All references listed in this application are incorporated in whole by reference for all purposes, as long as they do not conflict with the invention. While specific embodiments and examples of the disclosed subject matter have been discussed herein, these examples are illustrative and not restrictive. Many variations will become apparent to those skilled in the art upon review of this specification and the claims below.

1. A gene variant array comprising a plurality of gene products associated with one or more diseases arrayed on a substrate, wherein each discrete location of the array comprises a target gene product and gene product variants.

2. The array of claim 1, wherein each discrete location comprises a single target gene product and one or more gene product variants.

3. The array of claim 1, wherein the plurality of gene products are immobilized at each discrete location as expressed proteins.

4. The array of claim 1, wherein the plurality of gene products are expressed in situ at each discrete location of the array by in vitro transcription and translation of target gene nucleic acids and nucleic acids of gene variants obtained from one or more biological samples.

5. The array of claim 1, wherein the substrate is selected from the group consisting of a slide, a microwell plate, and a nanowell plate.

6. A method of preparing the gene variant array of claim 1, the method comprising;
(a) providing a first substrate comprising one or more disease-associated biomolecules at one or more discrete locations in an array format; and
(b) providing a second substrate comprising an array of biosensors configured to capture the one or more disease-associated biomolecules, wherein the second substrate is in proximity to the first substrate and wherein the array of one or more disease-associated biomolecules is in alignment with the array of biosensors,
wherein the apparatus is configured to detect at least one disease-associated target biomolecule in a test sample.

7. The method of claim 6, wherein the gene variant array is configured to detect post translational modification of proteins in the array.

8. The method of claim 6, wherein the gene variant array is configured to determine kinetic rates of post translational modification.

9. A method of detecting the presence a target biomolecule in a test sample, the method comprising
(a) contacting one or more disease-associated biomolecules to one or more discrete locations in an array format on a first substrate having at least two physically isolated regions;
(b) capturing the one or more disease-associated biomolecules at one or more discrete locations on a second substrate to form a monolayer of captured biomolecules in an array format on the second substrate, wherein the second substrate comprises an array of biosensors that capture the one or more disease-associated biomolecules;
(c) contacting a test sample to the array of captured biomolecules under conditions that promote binding of target biomolecules to the captured biomolecules if present in the test sample; and
(d) detecting binding of target biomolecules to the captured biomolecules at one or more discrete locations on the second substrate, wherein detectable binding indicates the presence of the target biomolecule in the test sample.

10. The method of claim 9, wherein the one or more disease-associated biomolecules are proteins expressed by in vitro transcription and translation (IVTT).

11. The method of claim 9, wherein the array of biosensors on the second substrate is aligned with the array of one or more disease-associated biomolecules, whereby one or more disease-associated biomolecules is captured directly onto active areas of corresponding biosensors on the second substrate.

12. The method of claim 11, wherein the active area of a biosensor is at least one surface in close proximity of a sensor device.

13. The method of claim 9, wherein at least a portion of biosensors of the array comprise an electrochemical sensor array, a metal or semiconducting surface, or an insulator surface.

14. The method of claim 9, wherein the biosensors comprise quantum dots, nanoparticles, beads, magnetic particles and wherein detection comprises optical detection.

15. The method of claim 9, wherein the biosensors comprise calorimetric sensors, potentiometric sensors, SERS (Surface Enhanced Raman Spectroscopy) sensors, amperometric sensors, conductometric sensors, ion channel sensors, ion sensitive sensors, impedance spectroscopy based sensors, or surface-plasmon-polariton sensors, or a combination thereof.

16. The method of claim 9, wherein the one or more disease-associated biomolecules are proteins that bind to the second substrate within about 1 nm to about 1 mm of the biosensors.

17. The method of claim 9, wherein the one or more disease-associated biomolecules are proteins that bind to directly to at least a portion of a biosensor surface.

18. The method of claim 17, wherein the proteins bind using a chemical tag, affinity tag, or covalent binding.
